(19) United States
(12) Patent Application Publication
(10) Pub. No.: US 2008/0090895 A1
(43) Pub. Date: Apr. 17, 2008

(54) Novel Pharmaceutical Composition And Their Uses Thereof For Controlling The Different Forms Of Addiction To Drugs

(75) Inventor: Mario Sanchez, Paris (FR)

Correspondence Address: BANNER & WITCOFF, LTD., 1100 13th STREET, N.W., SUITE 1200, WASHINGTON, DC, 20005-4051, US

(73) Assignee: Trimaran Limited, St. Albans (GB)

(21) Appl. No.: 11/666,981

(22) PCT Filed: Nov. 7, 2005
(86) PCT No.: PCT/FR05/02775

§ 371 Date: Oct. 24, 2007

(30) Foreign Application Priority Data
Nov. 5, 2004 (FR) 0411810

(51) Int. Cl. A61K 31/40; A61K 31/135; A61P 25/36

(52) U.S. Cl. 514/428; 514/648

(57) ABSTRACT

The invention relates to the necessaries of life, especially the field of therapeutics. The invention specifically relates to pharmaceutical compositions for helping users of addictive drugs to stop, said compositions being in the form of a combination of two medicaments consisting of a partial or full antagonist of dopaminergic receptors, especially receptors D2 and D3, and a prodopaminergic product, for oral, parenteral or transdermic administration. The invention also relates to method for controlling the different forms of addiction to legal or illegal drugs.

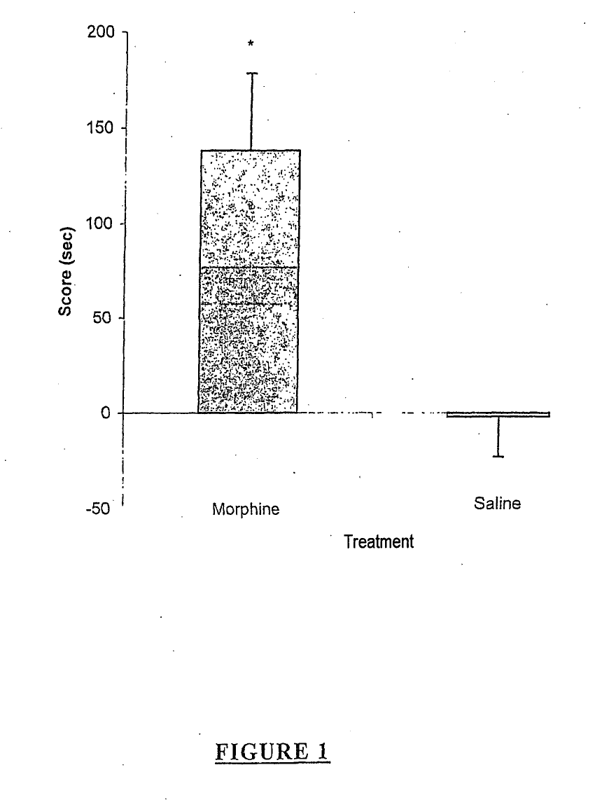

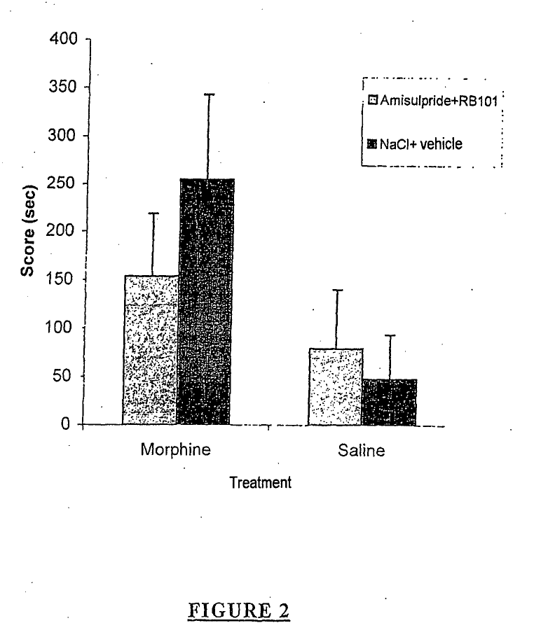

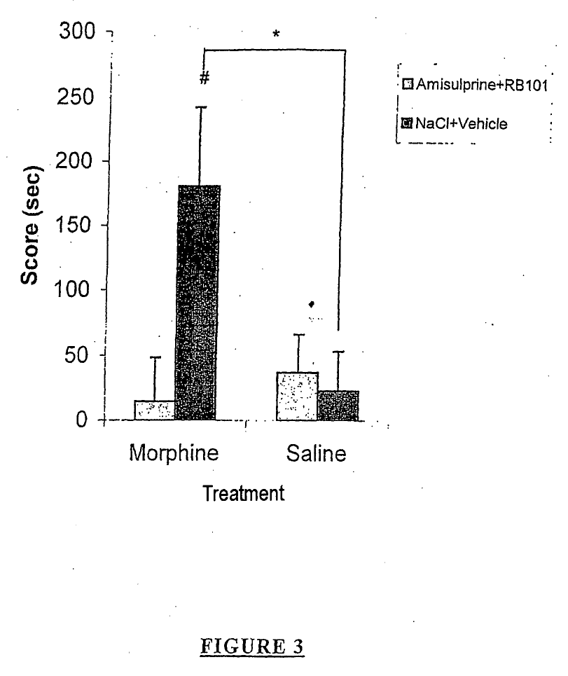

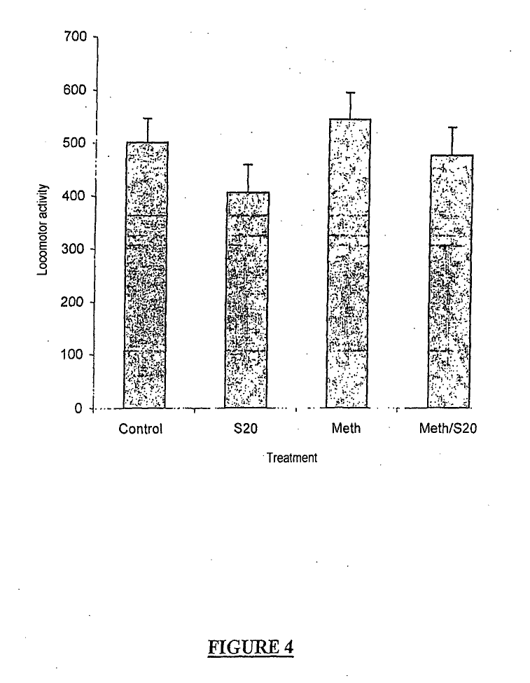

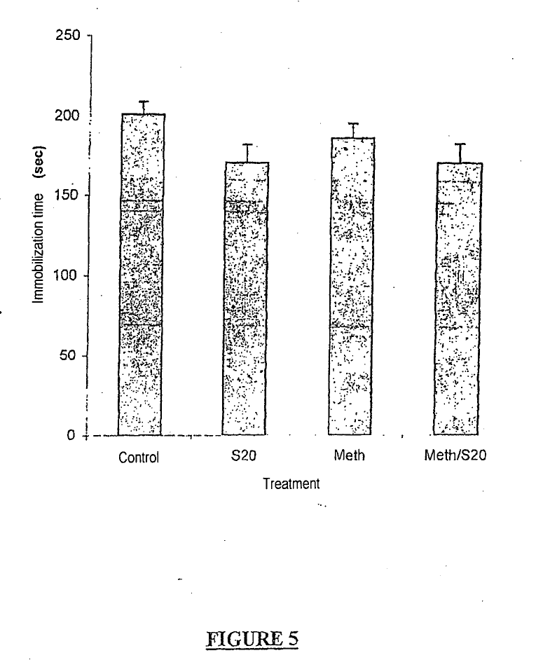

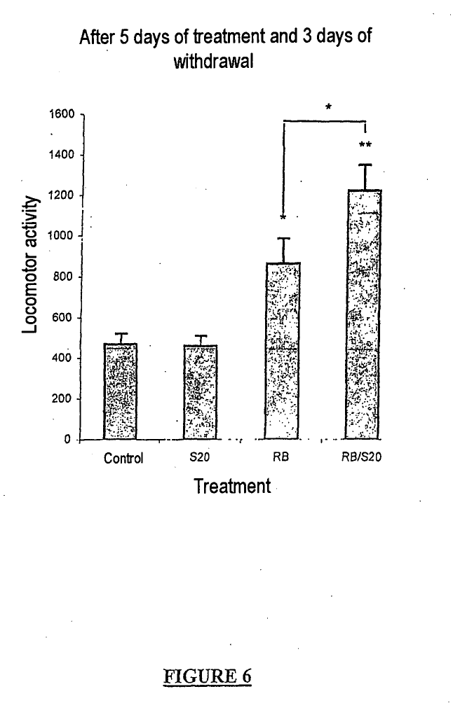

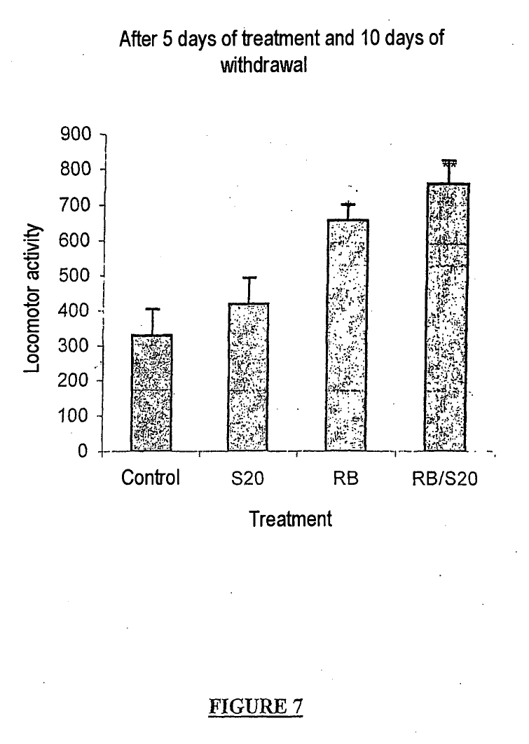

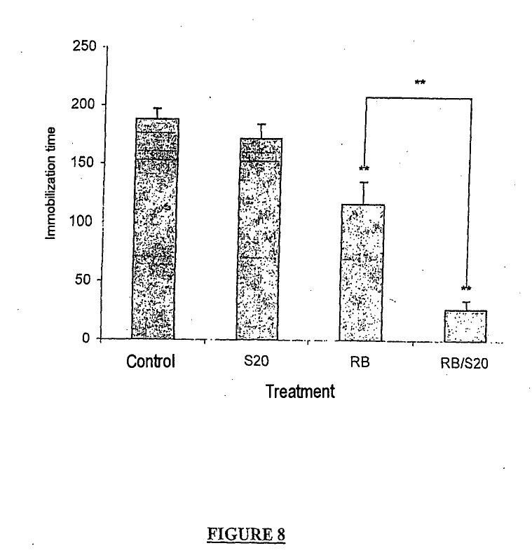

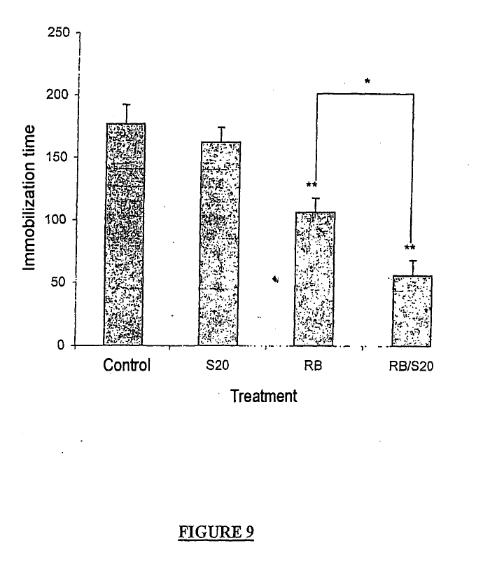

The invention relates to the field of life needs and more particularly to the therapeutic field.

The invention relates more particularly to pharmaceutical compositions for helping, in a powerful manner, the takers of addictive drugs to return to abstinence and thus to lead them towards regaining normal social and/or professional activity.

Addiction (or dependency) may be defined as a behavioral disorder, characterized by a compulsive search for the product that causes this dependency, despite the harmful consequences on the health, family, professional life, etc. of which the dependent person is fully aware.

This loss of behavioral control appears as a result of repetitive consumption, but, in the case of heroin and opiates, the transition from abuse of these substances to addiction may be very brief. This depends on a certain number of genetic and environmental parameters intrinsic to each individual.

This dependency is due to the excessive and repeated stimulation of the opioid receptors, in particular of mu type (Mattes et al. Nature, 1996, 383, 819-823), more particularly in the cerebral structures forming the limbic system (ventral tegmental area, nucleus accumbens, amygdala, prefrontal cortex, etc.). Changes gradually follow in the functioning of the neurons, which maintain this dependency and, most especially, induce a very powerful and very long-lasting remnance of the effects of the substance.

These effects are characterized by sedation, euphoria or a reduction in the inner tensions of the consumer. Furthermore, there is an “orgasmic” pleasure effect, known as a “rush”, which follows, for example, the injection of heroin. The effect of this substance and of opioids, or other highly addictive drugs such as cocaine, leads, on coming down, to overexcitment of the neuronal control systems that produce an inverse effect, i.e.: anxiety, dysphoria, etc. This inverse effect appears in particular during the stopping of consumption of the drug: this is the “withdrawal syndrome”, which is very painful and, in most cases, short-lived, giving rise to repetitive relapses.

One of the ways of reducing these very painful states that “hook” the dependent person on his drug is to seek to stabilize the patient by avoiding the ecstatic “rush” states, and by treating the cause of the major behavioral disorders that led to the addiction.

The most spectacular successes have been obtained by substituting heroin, or other addictive opioids, with substances that are also capable of stimulating the opioid receptors, but less powerfully, and for different reasons. For some of them, it is a problem of pharmacokinetics that leads to a long, slow impregnation of the brain with this opioid substance. As a result, the receptors are never powerfully stimulated, as they are with heroin, but they are stimulated sufficiently for the patient not to suffer from a lack and from an uncontrollable urge to obtain the “substance” (craving). This is the case for methadone, a full agonist, which has been used as a heroin substitution treatment since 1964 in the USA, and was approved by the FDA in 1973. Another substance that is increasingly used is buprenorphine, which is a partial agonist of the mu opioid receptors with a long duration of action. As a result, buprenorphine is incapable, even at high doses, of producing the “rush” described previously.

These substitution treatments give noteworthy results, but suffer from a major defect. They lead only to a mediocre reduction in addiction and, consequently, heroin-dependent individuals are often treated for years (up to 20-30 years), for example with methadone. This then amounts, as it were, to a dependency on the substitution substance.

Obviously, the ideal situation would be to find a treatment that significantly facilitates access to abstinence. However, the neurotransmitter involved in the euphoric effects of opioids is dopamine, which is released by the dopaminergic nerve endings, in particular in the nucleus accumbens and the prefrontal cortex. Dopamine interacts with the D1, D2 and D3 receptors, essentially to lead to the hedonic effect.

Blocking of these receptors with neuroleptics is very frequently used in certain major disorders, such as schizophrenia, panic attacks or generalized anxiety. This type of treatment generally leads the patient to a dysphoric state with reduction of the hedonic effects and of social activities.

The invention that is the subject of the present patent application lies in the fact that, against all expectation, the treatment of individuals dependent on heroin and opioids, but also, to a lesser extent, on psychostimulants (for example cocaine), with an antagonist of the dopaminergic receptors, in particular of the D2 and/or D3 type, leads to a rapid improvement in the state of inattention leading to the compulsive search for the addictive substance.

However, this significant improvement is obtained only when these antagonists are administered simultaneously or in combination with a substitution product (methadone, buprenorphine, LAM (Levo-alpha-acetylmethadol)) or all the other substances claimed for having this property of acting on the opioid receptors.

Thus, the combination, during the administration of the two substances (a dopaminergic antagonist and a prodopaminergic product) is capable of producing an anti-addiction effect, at least during the first weeks of treatment.

Without this finding being fully explained, it is the fruit of experimental clinical studies.

The improvement in the physical state of the dependent individuals is such that it very rapidly allows the establishment of a search for the underlying causes of the compulsive behavior, characteristic of the addiction.

One subject of the invention is thus, specifically, a pharmaceutical composition containing a combination of two medicaments, preferably in kit form, intended to be administered, simultaneously or successively, for facilitating withdrawal, which consists of a combination of a partial or full antagonist of the dopaminergic receptors, in particular of the D2 and D3 receptors, and of a pro-dopaminergic product, preferably an opioid substitution product, in the form of a pharmaceutical composition for oral, parenteral or transdermal administration.

The dopaminergic antagonist is preferably an antagonist of the D2 type, or most especially a D2/D3 antagonist.

Among the dopaminergic antagonists, mention may be made of pure dopaminergic antagonists and partial dopaminergic antagonists, also manifesting a serotoninergic component. Among the dopaminergic antagonists, the molecules most widely used are:
amisulpride,
risperidone,
the D3 antagonist known as SB 277011-A, described by Votel et al., in J. Neuroscience 22 (2002) 9595-9603.

Other dopamine antagonist substances, such as sulpiride, metoclopramide, or even olanzapine or haloperidol, may also be used.

The pro-dopaminergic product may be defined as a substance capable of binding to or in the opioid receptors, which only weakly manifests euphoric activity and/or which manifests only a limited addiction effect. Mention may be made in this regard of methadone, buprenorphine, the product known as LAM, nalorphine, naltrexate, Levallorphan and, in general, any substance described as having such a property.

The invention thus lies in the administration of such a combination either simultaneously in the form of a single defined pharmaceutical composition, or in the form of a kit containing each of said active principles in a separate form, which may thus be administered at variable dosages, or at different rhythms or in a different order, or in different forms.

The combination of the two active principles may thus be administered in two identical pharmaceutical forms [for instance plain tablets, gel capsules, sugar-coated tablets or drops], or in different forms.

The concentrations of active principles may also vary, passing from a high dose to a lower dose, as a function of the therapeutic needs, the pursuit of the treatment and the occurrence of side effects.

It is already known practice to use amisulpride or salts thereof, and especially S-(−)-amisulpride for the treatment of affective or cognitive symptoms of schizophrenia, for the treatment of autism or for the treatment of neuroleptic-induced tardive dyskensia (PCT/EP99/05325). Patent PCT/EP99/05325 also mentions that S-(−)-amisulpride may be used to counter “drug addiction” without any other details.

Amisulpride is one of the many representatives of the benzamide series described in U.S. Pat. No. 4,401,822 as an anti-apomorphine substance. The synthesis of amisulpride in racemic or enantiomerically pure [S(−)] form is described in patent application PCT/EP99/05325, along with that of salts thereof.

Amisulpride is described in pharmacology as displacing [³H]-raclopride from the limbic D2 receptors. Amisulpride also manifests an antagonist action against apomorphine. As a result of its central action, amisulpride may be considered as an antipsychotic medicament in the case of individuals suffering from schizophrenia, most especially by manifesting fewer side effects than the known antipsychotic neuroleptic products, such as extrapyramidal syndrome, etc.

Amisulpride is thus a known medicament, which has been used hitherto in other neuropsychiatric indications.

The anti-addictive effect sought in the present invention is another antagonist effect with respect to the dopaminergic receptors, especially the D2 and D3 receptors.

The effect of the medicaments that are the subject of the present combination appears rapidly and, already in preclinical studies, a positive effect is noted taking into account the impregnation effect.

The doses administered in the context of the pharmaceutical compositions according to the invention will be variable as a function of the desired effect, the length of existence of the dependency on the addictive drugs and the intensity of the action against the desired addiction.

The doses of anti-dopaminergic substance may range from 1 mg to 1200 mg per single intake. The doses of pro-dopaminergic substances, rising up to a plateau, will range from 0.2 mg to 300 mg.

In one preferred embodiment of the invention, the combination will be formed from tablets of anti-dopaminergic substance, such as amisulpride, containing from 400 mg to 1200 mg of active principle, and of tablets of pro-dopaminergic substance, for instance buprenorphine, at a dose of from 0.2 mg to 30 mg per single intake. The doses of pro-dopaminergic substance will be higher in the case of rapid metabolizers who thus tolerate higher doses (200 to 300 mg).

Another embodiment that is particularly useful will be the presentation in the form of a kit containing, for example, two bottles of a solid or liquid preparation, one of the bottles containing a solution of anti-dopaminergic substance, the other bottle containing a solution or a suspension of substitution substance, for instance an aqueous methadone suspension or syrup.

In another embodiment of the combination according to the invention, combined forms may be produced, especially dry forms containing the two active principles and thus achieving a simultaneous administration. Two-coat plain tablets or two-core sugar-coated tablets containing, in one of the parts of the pharmaceutical form, the anti-dopaminergic substance, and, in the other part, the pro-dopaminergic substance, may thus be envisioned. Scored tablets are also an easy administration form.

Injectable forms may also be prepared. They allow the simultaneous administration of the two active principles of the combination. They are justified in particular for preparing deposit forms with sustained action. Transdermal forms may also be envisioned with a sustained effect.

It is also possible to prepare fixed combinations containing determined doses of each of the active principles either in free form, or in physically combined form, or in chemically combined form, for instance a double salt with a polycarboxylic acid, or with an acidic resin. These fixed combinations are, however, less easy to use, since they do not allow the dosage to be modified. They are, however, useful, especially at the start of treatment, for determining the sensitivity of the patient and for monitoring the absence of side effects or the more or less rapid appearance of the benefit of the anti-dopaminergic effect.

The usual dosage regimen generally consists in using low doses of pro-dopaminergic medicament, and then in gradually increasing the doses to obtain a “plateau” effect.

In the case of amisulpride, the daily dosage will range from 400 to 1200 mg, with a single intake of 100 to 400 mg.

In the case of risperidone, the dosage will range from 1 to 16 mg per day.

The administration of pro-dopaminergic products, and especially of methadone, will range from. 5 to 60 mg per dosage intake. The doses of buprenorphine, morphine sulfate or nalorphine will be of the same order of magnitude.

The order of administration of the two components of the combination according to the invention is not a determining factor and may be modified according to the therapeutic needs. It appears preferable to ensure the administration firstly of the pro-dopaminergic substance, and then of the anti-dopaminergic product. It is possible, on the other hand, to administer the anti-dopaminergic product first, then followed by administration of the pro-dopaminergic product. In any case, it is more convenient for the administration of the two active principles to be simultaneous.

A subject of the invention is also a pharmaceutical composition consisting of a combination of an anti-dopaminergic product or a salt thereof, and of buprenorphine, containing, for example, from 400 to 1200 mg of amisulpride and from 0.2 to 30 mg of buprenorphine in an inert, nontoxic, pharmaceutically acceptable excipient or vehicle, with the dosage being modified, firstly increasing, and then, when the threshold effect is achieved, the dosage is reduced.

Another subject of the invention lies in the production of a kit containing a first pharmaceutically suitable dosage of anti-dopaminergic substance in base form or in salt form, in racemic form or in enantiomeric form, at a dose of from 100 to 400 mg, and a second pharmaceutically suitable dosage of methadone containing from 5 to 60 mg of methadone per single intake.

The invention also relates to an anti-addiction medicament consisting of the combination of sulpiride in racemic or optically active form, and in free form or salified with a mineral or organic acid, and of buprenorphine.

The combination according to the invention is intended to be administered at a rate of one to four times a day, at predetermined intervals, to ensure constant impregnation of the individual with medicament.

The pharmacological and clinical trials, the details of which are given in the appendix, show the efficacy of the combination according to the invention.

The invention also relates to a method for combating the different forms of addiction to licit or illicit drugs, which consists in administering to individuals displaying phenomena of addiction to the illicit drugs a sufficient and effective amount of a combination of a pro-dopaminergic agonist and of a dopaminergic antagonist simultaneously, in a single or separate pharmaceutical form, or alternatively in batch form, by first administering the dopaminergic agonist, in a given pharmaceutical form, followed by the dopaminergic antagonist in another pharmaceutical form, for example in kit form.

The method described above is most particularly suitable for combating addiction to opiate drugs, for instance heroin. It also finds a use in combating the use or abuse of active principles that lead to addiction, for instance amphetamine and derivatives thereof, alcohol, cocaine and NMDA.

### EXPERIMENTAL SECTION

### 1. Opiates and the Opioid System

1.1 The Opioid Receptors

Activation of the opioid receptors produces a wide variety of physiological and pharmacological responses. Specifically, the opioid system is involved especially in modulating stress, pain, mood, the cardiovascular function, and the taking of food (Vaccarino et al., 2000).

The use of radiolabeled ligands with high specific activity has allowed the discovery, in the central nervous system of mammals, of stereospecific, saturable and high-affinity receptors. These specific membrane binding sites for exogenous opiates were demonstrated by three teams (Simon et al., 1973; Terenius, 1973; Pert and Snyder, 1973). More recently, the receptors have been cloned and are defined as being of three types: ε, μ and κ (Kieffer et al., 1992; Chen et al., 1993; Yasuda et al., 1993). Depending on their sequence, it clearly appears that the opioid receptors belong to the major family of seven-domain trans-membrane receptors binding the heterotrimeric G proteins (Dohlman et al., 1987). These receptors have 60% sequence homology in man, the most conserved sequences being the transmembrane domains and the intracellular loops. Furthermore, they are differently distributed in the central nervous system. The μ opioid receptors are widely present throughout the central nervous system, with very high concentrations in certain regions such as the basal ganglions, the limbic structures, the thalamic nuclei and regions important for nociception. The delta and kappa receptors have a more reduced distribution, and are most especially present in the ventral and dorsal striatum for the former, and in the dorsal striatum and the preoptic area for the latter (Mansour et al., 1988).

The signal transduction cascades associated with the opioid receptors have been widely studied in various tissues, cell types or neuronal preparations. It has been shown that these three receptors are coupled to the Gi/Go proteins which modulate numerous effectors. Specifically, the opioid receptors inhibit adenylate cyclase activity (Sharma et al., 1977), thus leading to a reduction in the level of intracellular cAMP, reduce the calcium conductance (Hescheler et al., 1987; Surprenant et al., 1990), stimulate the potassium channels (North et al., 1987) and increase the level of intracellular calcium (Jin et al., 1992). More recently, it has been shown that these receptors are capable of generating mitogenic signals by activating the MAP-kinase pathways (Fukada et al., 1996).

1.2 The Endogenous Opioid Peptides

The endogenous ligands of the opioid receptors are the endomorphines (Hughes et al., 1975). These are neuropeptides released into the synaptic space, from large vesicles with a dense core, as a consequence of stimulation of neurons where they coexist with other neurotransmitters. Endomorphines are derived from distinct precursors and are heterogeneously present in the various populations of neurons of the central nervous system. Proopiomelanocortin (or POMC) gives rise to P-endorphin and to related peptides, pro-enkephalin A is the source of the enkephalins (Met- and Leu-enkephalin) and of similar peptides, and prodynorphin gives rise to the neo-endorphins and to dynorphin (Akil et al., 1998).

1.3 Enkephalin Degradation Enzymes and Synthetic Inhibitors of these Enzymes

Enkephalins have a very short lifetime after their release (less than a minute). This brevity is not due, as for most of the standard neuromediators, to a reuptake system, but to their enzymatic degradation. Met-enkephalin (Try-Gly-Gly-Phe-Met) and Leu-enkephalin (Tyr-Gly-Gly-Phe-Leu) are rapidly hydrolyzed by cleavage of the Gly-Phe bond with a peptidase that was initially known as enkephalinase, which has since been demonstrated to be identical to neutral endopeptidase (NEP), and at the Tyr-Gly bond with aminopeptidase N (APN) (Roques, 1986). These two enzymes belong to the same group of zinc metallopeptidases.

Many inhibitors of these enzymes have been synthesized in order to increase the lifetime of enkephalins, and thus to prolong (their effects (Roques, 1993). However, in order to fully protect the endogenous opioid peptides from enzymatic degradation, it is necessary to inhibit not only NEP but also APN (Bourgoin et al., 1986).

Several series of mixed enkephalin inhibitors have been developed (Roques, 1986), including RB101, which is a molecule capable of crossing the blood-brain barrier (Fourni-Zaluski et al., 1992), but which is also endowed with low oral bioavailability.

Enkephalin catabolism inhibitors increase the extracellular concentration of enkephalins without affecting their release (Daugé et al., 1996; Bourgoin et al., 1986; Waksman et al., 1985). The advantage of these molecules is that, even at very high doses, they never induce pharmacological responses that are as powerful as that of morphine (Ruiz-Gayo et al., 1992; Abbadie et al., 1994), and are thus free of the standard side effects of opiates (constipation, dryness of the mouth, itching, irregular periods and, more seriously, gastrointestinal disorders and respiratory depression).

1.4 Opiates

The exogenous opioid receptor ligand that has been known for the longest time and that is used in medicine is morphine, an alkaloid derived from Indian poppy.

Other substances have the same pharmacological characteristics as morphine. Heroin (diacetylmorphine, diamorphine), which is metabolized to morphine, was introduced into medicine in 1898 in the treatment of tuberculosis. Nowadays, this substance is highly favored by drug addicts, due to its rapid penetration into the brain where it generates an orgasmic response, the “high”.

Other opiate agonists are nowadays used in substitution treatments: this is the case for methadone and buprenorphine. Methadone is a synthetic opiate and, like morphine, is a preferential agonist of the μ receptors.

Other synthetic opioids such as DAMGO and DPDPE are conventionally used as selective ligands of the μ and δ receptors, respectively, in experimental pharmacology (Handa et al., 1981; Mosberg et al., 1983).

Another class of exogenous opioid receptor ligands exists: the opioid antagonists. Mention may be made, inter alia, of naloxone, which is used therapeutically in the treatment of acute opiate intoxication. This molecule binds with the same affinity to the two μ and δ receptors. Another known antagonist is naltrindole, which binds with very strong affinity to the δ receptors (Fang et al., 1991). It is widely used in experimental pharmacology.

### 2. Opioid Addiction

2.1 Introduction: Dependency or Addiction

According to the WHO definition, dependency/addiction is a syndrome in which the consumption of a product becomes a requirement greater than that of other behaviors that were previously of maximum importance. Dependency becomes established with repetition of the taking of drugs and is characterized by a compelling need for the drug, which leads to its compulsive search. Dependency has two facets: physical and psychic.

The physical component obliges the drug addict to consume the drug at the threat of experiencing pain specific to the withdrawal syndrome (which, apart from exceptional cases, is not mortal, despite the strength of the pain experienced). It may disappear after a few days. The psychic component is the drug addict's desire to recommence, and is associated with a strong stimulation of the brain by the reinforcement/reward system and is the cause of many relapses in drug addiction. It may last for several years.

2.2 Opiate Dependency and Tolerance

Tolerance is the process of adaptation of a body to a substance, which is reflected by the gradual attenuation of the effects of said substance, and results in the need to increase the dose in order to obtain the same effects. In animals, tolerance results in a decrease in the behavioral effects induced by the drug following its repeated administration.

Chronic activation of the opioid system by exogenous ligands such as morphine leads to the establishment of a dependency characterized by the compulsive search for the drug. In animals, especially in rats, many experimental models have made it possible to demonstrate the behavioral effects of opiates. Techniques, such as self-administration or the conditioned preference of place, have demonstrated the reinforcing effects of heroin and morphine (McBride et al., 1999), which effects appear to be mainly mediated by the μ opioid receptors (Matthes et al., 1996).

2.3 Withdrawal

The abrupt interruption of consumption of drugs is manifested, in drug addicts, by physical and psychic symptoms. Withdrawal from opiates is manifested, inter alia, by hypertension and abdominal cramps, but also by anhedonism and dysphoria.

In animals, withdrawal from opiates may be brought about by the administration of an opioid antagonist, naloxone. Several behavioral changes are then observed in morphine-dependent rats: increase in grooming, mastication, blinking of the eyes, but also diarrhea or weight loss.

3. Dopaminergic System and Amisulpride

3.1 The dopaminergic System

Dopamine acts on two classes of receptors: “D1-like” and “D2-like”. The D1-like receptors (D1 and D5) are coupled via Gs to adenylate cyclase and allow the production of cAMP, which triggers numerous metabolic responses dependent on protein kinase A. The D2-like receptors (D2, D3 and D4) are coupled to Gi/o and inhibit the synthesis of cAMP, which in particular facilitates opening of the hyperpolarizing K⁺ channels.

The dopamine neurons are mainly assembled in two mesencephalic nuclei. One is the ventral tegmentum or ventral tegmental area (VTA, or mesencephalic area A10), whose axonal projections innervate the cortex (especially in its anterior part), the limbic system (especially the septum and the amygdala) and basal nuclei (putamen and nucleus accumbens). The majority of these fibers pass through the median telencephalic fascicle (MTF) and are involved in the processing of cognitive-affective information.

In point of fact, this neuronal cabling belongs to the reinforcement/reward system, which produces a very strong cerebral stimulation in order to evoke pleasure (hedonic action) during behaviors essential to the survival of the species or of the individual. It is this motivation circuit that is bypassed by drugs. Thus, by producing pleasure, drugs motivate the individual towards compulsive behavior where drug use replaces the survival behaviors.

The other dopaminergic nucleus is the substantia nigra (locus niger or mesencephalic area A9) that emits axons toward the striatum (caudate nucleus and putamen) and that participates in controlling locomotion. Drugs that modify the level of release of dopamine in the striatum disrupt motor functions.

3.2 Dopamine-Dependent Mechanisms

The administration of morphine stimulates the activity of the dopaminergic neurons in the substantia nigra and in the VTA, which leads to an increase in dopamine release into the caudate nucleus-putamen and into the nucleus accumbens (Matthews and German, 1984; Spangel et al., 1990; Di Chiara and North, 1992).

It is commonly accepted that this increase is due to an indirect action of opioids. Specifically, activation of the μ receptors present at the surface of the GABAergic interneurons located in the reticular substantia nigra and the VTA are believed to lead to removal of the inhibition exerted by these interneurons on the dopaminergic neurons (Johnson and North, 1992; Bontempi and Sharp, 1997).

3.3 Amisulpride, a Dopaminergic Antagonist

Amisulpride is a molecule chemically related to benzamides. At low doses, amisulpride has an antagonist effect on the D2 and D3 presynaptic receptors (net effect: facilitation) of the frontal cortex. In contrast, amisulpride used at high doses inhibits the post-synaptic D2 and D3 receptors (net effect: blockage) on the limbic system. Furthermore, it is free of extrapyramidal effects, since it has only low activity on the striatum (Perrault et al., 1996). All these factors make this molecule an atypical antipsychotic, which is nowadays used in the treatment of the positive and negative symptoms of schizophrenia.

### Materials and Methods

### 1. Animals and Treatments

The animals used in this study are male mice of OF1 strain weighing about 20 g at the start of the experiments (Charles River, France). They live in an environment whose daily lighting cycle (07:30 h; 17:30 h) is constant throughout the year, and the temperature is maintained at about 22° C. The mice have free access to water and food, and the experiments are performed in accordance with the international rules on ethics in animal experimentation.

The animals are treated chronically via the intraperitoneal route (IP) with amisulpride or physiological saline. The injections are performed twice a day, with an interval of about eight hours between each administration, over a period ranging between five days and three weeks. On the day of the pharmacological test, the animals do not receive any amisulpride. RB101 is administered on the day of the test intravenously (IV), 10 minutes before the start of the test (except for the measurements of locomotor activity performed immediately after injection).

### 2. Products

The amisulpride (injectable solution at 200 mg/4 mL) is used in the form diluted with physiological saline.

RB101 is a synthetic product described by Baamonde et al. Europ. J. Pharmacol. (1992) T 216, pp. 157-166. RB101 is dissolved in an ethanol (10%)/Cremophor EL (10%)/distilled water (80%) vehicle.

Methadone hydrochloride and morphine hydrochloride are commercial products. They are dissolved in physiological saline.

### 3. Methods

3.1 Measurement of the Locomotor Activity

The mice are placed individually in a sound-insulated plastic cage (255 cm×205 cm) and are exposed to a light intensity of 5 lux. The animals' movements are captured by photoelectric cells for 45 minutes and recorded by computer. The animals receive the vehicle (ethanol (10%)/Cremophor EL (10%)/water (80%)) or RB101 (5 mg/kg) intravenously at a volume of 0.1 ml/10 g. The experiment starts immediately after injection of the product. In this study, the term “locomotor activity” takes into account only the horizontal movements of the animals.

3.2 Measurement of the Analgesia by the Hotplate Test

The mice are placed individually inside a cylinder, on a plate heated to 52±1° C. by a water circuit. The lag time for the jumping of the mice is measured, the value 100 of the percentage of analgesia corresponding to a time limited to 240 seconds in order to avoid skin lesions. The test is performed 10 minutes after injection of RB101 (5 mg/kg, IV) or of the vehicle. The results are expressed as a percentage of analgesia calculated by means of the following formula: (mean of the jumping lag times for the treated group—mean of the jumping lag times for the control group)/(240−mean of the jumping lag times for the control group)×100. The results are expressed as mean ±sem.

3.3 Test of Forced Swimming (Porsolt's Test): Model of Depression

The mice are placed individually in a cylindrical container filled to a height of 15 cm with water, the water being at room temperature. After a delay of 2 minutes, the total immobile time of the animal is measured for 4 minutes. The movements necessary for the animal to keep its head above water are not counted.

3.4 Conditioned Preference of Place: Measurement of the Psychic Dependency

The apparatus for conditioned preference of place consists of a box divided into three separate compartments: a black compartment with a smooth floor, a black and white stripy compartment with a rough floor, and a neutral central compartment.

The Test Proceeds in 3 Phases:

a pretest phase: the animal is placed in the neutral central compartment and has free access to the three compartments of the apparatus for 20 minutes. The time spent in each compartment is recorded using a camera connected to a computer. The mice showing a spontaneous preference for one of the compartments (i.e. spending more than 75% of the allotted time in one of the side compartments) are removed from the experiment.

After this first session, the animals are randomized in order to assign a treatment to them (morphine or physiological saline, SC) and the compartment in which they will receive the drug (black or black and white stripy compartment). It is chosen to condition the animals in the compartment for which the “preference” is the least pronounced;
a conditioning phase: the animals receive alternatively morphine (10 mg/kg, SC) or physiological saline for three consecutive days, the physiological saline being injected in the morning and the morphine in the afternoon for the same animal. The animals are kept in one or other of the compartments for about 20 minutes, immediately after injection. The compartment associated with the drug is always the same for the same mouse;
a test phase: as for the pretest phase, the animals are placed in the central compartment and have free access to the three compartments. They receive no injection of morphine or of saline solution on that day.

The scores correspond to the difference between the times spent during the test phase and the pretest phase in the morphine-associated compartment.

### 4. Statistical Analysis

A one-factor (treatment) analysis of variance (ANOVA) is used for all the behavioral tests performed, followed by a Student-Newman-Keuls test if p<0.05 in the ANOVA. In all the cases, the significance is accepted once p<0.05.

### Results

### 1. Determination of the Doses of RB101 and of Amisulpride used

1.1 RB101 Dose Effect on the Hot Plate

The hotplate test is conventionally used to evaluate the analgesic power of molecules. This is a method involving a response to a central integration, the jumping being associated with a wish to flee from the painful stimulus. The analgesic power of RB101 in this test has been shown previously (Noble et al., 1992), and a dose effect was demonstrated to start this study. Specifically, a search is conducted to find the dose of RB101 for which about 40% analgesia is obtained, which allows possible observation of a potentiation of its effects by amisulpride. Three doses were tested: 2.5 mg/kg, 5 mg/kg and 10 mg/kg intravenously, 10 minutes before the start of the test.

The 5 mg/kg dose allows an analgesia of 45.2%±10.6%. This is thus the dose adopted for the purpose of combination with amisulpride.

1.2 Determination of the Amisulpride Dose in Locomotor Activity

A molecule endowed with dopaminergic antagonist activity reduces locomotor activity. It is this property that is involved in order to determine the dose at which amisulpride has dopaminergic antagonist activity in mice (i.e. an effect on the D2 and D3 post-synaptic receptors, and not on the D2 and D3 auto-receptors). The doses tested are: 0.5 mg/kg, 2 mg/kg, 10 mg/kg, 20 mg/kg and 50 mg/kg.

The reduction in locomotor activity is significant at and above 10 mg/kg. The dose chosen is 20 mg/kg, for which dose the dopaminergic antagonist activity is manifest and indisputable.

2. Determination of the Duration of Treatment with Amisulpride (Amisulpride/RB101 Combination and Measurement of Locomotor Activity)

In contrast with amisulpride, RB101 alone induces an increase in locomotor activity in mice (Baamonde et al., 1992).

The treatment with amisulpride (20 mg/kg, IP, twice/day) was first performed for three weeks, after which RB101 (5 mg/kg, IV) was injected and the locomotor activity measured immediately after, for 45 minutes.

The effects of RB101 were significantly potentiated by amisulpride after three weeks of treatment. The question was thus posed as to whether this duration of treatment could be shortened. The locomotor activity was thus measured this time after only five days of treatment.

The potentiation of the effects of RB101 by amisulpride persists, even after only five days of treatment. It may thus be wondered how long this potentiation persists after stopping the treatment with amisulpride. The locomotor activity is thus measured after three days or ten days of stoppage of treatment, in mice that have received a treatment for five days according to the figure below:

[image]

The potentiation of the effects of RB101 after a five-day treatment with amisulpride is still present after three days, but no longer exists after ten days of withdrawal.

For the study, it was chosen to continue the experiments after a five-day treatment with amisulpride, and to perform the same experiments after three days of withdrawal in the case of success.

3. Amisulpride and RB101 Combination in the Forced Swimming Test

The forced swimming test is conventionally used to evaluate the antidepressant effect of molecules. RB101 alone is endowed with properties of antidepressant type (Baamonde et al., 1992), since it reduces the immobile time of mice in this test.

The amisulpride treatment is performed for five days, and RB101 is injected on the day of the test (the day after stopping the treatment).

There is also a potentiation of the effects of antidepressant type of RB101 (5 mg/kg, IV) by amisulpride after five days of treatment.

The same test is performed after three days of withdrawal.

The potentiation of the effects of antidepressant type of RB101 (5 mg/kg, IV) by amisulpride (five days of treatment) is still present after three days of withdrawal.

4. Amisulpride and RB101 Combination in the Hotplate Test

The amisulpride treatment is performed for five days, and RB101 (5 mg/kg, IV) is injected on the day of the test (the day after stopping the treatment).

RB101 has by itself an analgesic effect (38.4%±10.8%). The amisulpride/RB101 combination has a level of analgesia of 49.6%±8.9%. However, there is no significant difference between these two groups, and thus no potentiation of the effects of RB101 by amisulpride in the hotplate test (after five days of treatment).

5. Amisulpride/RB101 Combination and Conditioned Place Preference

In a first stage, the animals are conditioned in the manner described in the “Materials and methods” section. The results obtained after this conditioning are shown on graph I in the appendix, which shows the place preference conditioned with morphine (10 mg/kg SC, n=10 mice per group) * p<0.05 relative to the saline solution group.

Place preference is observed with morphine (10 mg/kg, SC), which clearly reflects the reinforcing effects of this drug.

The mice of the two morphine and physiological saline groups are then divided into two subgroups of equal size, one subgroup being treated with amisulpride according to the usual protocol for five days, the other subgroup receiving injections of physiological saline.

A second test is performed on these mice on the sixth day, the animals that have been treated with amisulpride receiving RB101 on the day of the test, the others receiving vehicle.

The results of this test are shown on graph II attached in the appendix. It shows the effects of the treatment with amisulpride at 20 mg/kg via the IP route twice a day for 5 days combined with RB 1M 55 mg/kg IV on the day of test 2) on the animals used previously in test I.

No group obtains a significant result relative the control group (physiological saline, NaCl+vehicle), although a tendency toward persistence of the place preference is observed in the case of the mice of the morphine group that have not received the amisulpride+RB101 treatment. The mice of the morphine group that have received this treatment themselves appear to resemble the mice of the physiological saline group.

In order to see whether this tendency persists or not, a third test is performed four days after the test. No RB101 is injected on the day of test 3.

The results obtained are given on graph III. They show the effect of the treatment with amisulpride (20 mg/kg via the IP route twice a day for 5 days)+RB101 (5 mg/kg via the IV route, on the day of test 2), 4 days after test 2, on the same animals.

p<0.05 relative to the morphine/amisulpride+RB1M group

p<0.05 relative to the control group

A place preference is found in the case of the mice of the morphine group that have not received the amisulpride+RB101 treatment. Furthermore, there is now no place preference in the case of the mice of the morphine group that have received the amisulpride +RB101 treatment.

6. Amisulpride and Methadone Combination: Measurement of Locomotor Activity

The amisulpride treatment is performed for 5 days, and methadone is injected on the day of the test (0.25 mg/kg, IV). The methadone dose chosen induces a level of analgesia comparable to that of RB101 in the hotplate test.

Graph IV shows the measurement of the locomotor activity after 5 days of amisulpride treatment (s20) (20 mg/kg via the IP route, twice a day). Methadone is injected on the day of the test, immediately before the start of the test (n=10 mice per group).

No group obtains a significant result relative to the control group.

7. Amisulpride and Methadone Combination in the Forced Swimming Test

The amisulpride treatment is performed for 5 days, and methadone is injected on the day of the test (0.25 mg/kg, IV).

Graph V appended below shows the measurement of the immobile time in the forced swimming test after 5 days of amisulpride treatment (S20) at a rate of 20 mg/kg via the IP route, twice a day.

Methadone (meth) is injected on the day of the test, 10 minutes before the start of the test (n=10 mice per group).

Graph VI shows the measurement of the locomotor activity after 5 days of amisulpride treatment (S24) at a rate of 20 mg/kg via the IP route, twice a day and 3 days of withdrawal. RB101 5RB) is injected on the day of the test, immediately before the start of the test (n=10 mice per group)

* p<0.05

** p<0.01 relative to the control group.

Graph VII illustrates the results obtained after 10 days of withdrawal.

Graph VIII shows the results obtained by measuring the immobile time in the forced swimming test in the case of mice that have received an amisulpride treatment ((S20) at a rate of 20 mg/kg via the IP route, twice a day) for 5 days. RB101 (RB) is injected 10 minutes before the start of the test (n=10 mice per group)

** p<0.001 relative to the control group.

Graph IX shows the results obtained by measuring the immobile time in the forced swimming test in the case of mice that have received an amisulpride treatment (S20) at a rate of 20 mg/kg via the IP route, twice a day for 5 days followed by 3 days of withdrawal. RB101 (RB) is injected 10 minutes before the start of the test (n=10 mice per group)

* p=0.005

** p<0.001 relative to the control group.

The results obtained in this study show that the effects of a mixed inhibitor of enkephalin catabolism, RB101, are potentiated in mice that have been pretreated chronically with amisulpride, a D2/D3 dopaminergic antagonist, in two of the three pharmacological tests performed (forced swimming test and measurement of the locomotor activity). It is interesting to note that this potentiation is obtained quite rapidly, since five days of amisulpride treatment suffice, and that this effect appears to persist over time (three days), even after this short duration of treatment.

Furthermore, since the same tests performed after a single injection of amisulpride and of RB101 do not allow this potentiation to be obtained, there is a need for “chronic” blocking, even of short duration, of these dopaminergic receptors in order to obtain greater stimulation of the opioid system with RB101.

The use of methadone instead of RB101 did not produce any potentiation of the effects of methadone when it is combined with a chronic amisulpride treatment in the locomotor activity and forced swimming tests. Furthermore, the combination of RB101 and of amisulpride in the hotplate test showed no potentiation.

These results may be explained by a preferential involvement of opioid receptors other than the μ receptors in this combination. Specifically, the hotplate test predominantly involves a supraspinal opioid analgesia mediated by the μ receptors (Roques, 1993). As regards methadone, it is a preferential agonist of the μ receptors. It has also been shown that the effects of antidepressant type of enkephalins, protected by RB101, were mediated by a stimulation of the δ rather than the μ opioid receptors (Baamonde et al., 1992). The contribution of the δ receptors toward improving mood has also been shown (Filliol et al., 2000).

It may thus be thought that the amisulpride/RB101 combination allows a potentiation of the effects of RB101 by preferentially acting on the 6 opioid receptors. It is also interesting to use a preferential 6 receptor antagonist, for instance naltrindole, and to see whether it is possible to block the effects obtained in the amisulpride/RB101 combination. A preferential 6 receptor agonist, for instance SNC 80 or BUBU (which may be administered systemically) may also be used, instead of RB101 after an amisulpride treatment, in order to observe its effects in the pharmacological tests used in this study (forced swimming test and measurement of the locomotor activity).

However, it should be pointed out that at the chosen dose (0.25 mg/kg, IV), methadone alone did not show a significant effect in locomotor activity and in forced swimming, whereas, at this same dose, it induces pronounced analgesia in the hotplate test.

However, methadone is known to have hyperlocomotor activity in mice (Browne, 1980), and has, as an opioid agonist, effects of antidepressant type. It should be confirmed that the absence of potentiation of the effects of methadone (0.25 mg/kg, IV) by amisulpride obtained in this study is not due to the use of an excessively low dose of methadone, by repeating the tests performed over a higher range of doses (the risk then being that of affecting only the μ opioid receptors if excessively high doses are used).

The existence of potentiation of the effects of RB101 by amisulpride in the tests of forced swimming and of measurement of locomotor activity, although this potentiation is not found in the hotplate test, may also be explained by the existence of regioselectivity of action of amisulpride. Specifically, the locomotor activity and the effects of antidepressant type are behaviors strongly involving the dopaminergic pathways, whereas the analgesic effects are most especially due to the opioid system.

Cerebral microdialysis experiments may also be performed, to allow an evaluation of the extracellular levels of enkephalins obtained in different regions of the brain (for example the nucleus accumbens and the striatum, which form part of the limbic system, and also the periaqueducal gray matter, which is more particularly involved in pain) after chronic treatment with amisulpride.

In a context of dependency, represented in the study by the model of conditioned place preference, it appears that the chosen treatment protocol (amisulpride for five days, RB101 on the sixth day) suppresses the expression of place preference in the dependent animals, five days after stopping the amisulpride treatment (test 3). However, it should be pointed out that the test performed the day after stopping the treatment (test 2) does not allow a significant result to be obtained. These results thus indicate that the amisulpride/RB101 combination might be effective in the context of heroin dependency, although the protocol used can still be improved, and the results obtained herein confirmed.

### BIBLIOGRAPHY

1. Abbadie C, Honore P, Fournie-Zaluski M C, Roques B P, Besson J M. Effects of opioids and non-opioids on c-Fos-like immunoreactivity induced in rat lumbar spinal cord neurons by noxious heat stimulation.
Eur J Pharmacol. 1994 Jun. 13; 258(3):215-27.
2. Akil H, Owens C, Gutstein H, Taylor L, Curran E, Watson S. Endogenous apioids: overview and current issues.
Drug Alcohol Depend. 1998 Jun.-Jul.; 51(1-2):127-40.
3. Baamonde A, Dauge V, Ruiz-Gayo M, Fulga I G, Turcaud S, Fournie-Zaluski M C, Roques B P. Antidepressant-type effects of endogenous enkephalins protected by systemic RB 101 are mediated by opioid delta and dopamine D1 receptor stimulation.
Eur J Pharmacol. 1992 Jun. 5; 216(2):157-66.
4. Baik J H Picetti R, Saiardi A, Thiriet G, Dierich A, Depaulis A, Le Meur M Borrelli E. Parlinsonian-like locomotor impairment In mice lacking dopamine D2 receptors.
Nature. 1995 Oct. 5; 377(6548):424-8.
5. Bontempi B, Sharp FR Systemic morphine-induced Fos protein in the rat striatum and nucleus accumbens is regulated by mu oploid receptors in the substantia nigra and ventral tegmental area.
J Neurosci. 1997 Nov. 1; 17(21):8596-612.
6. Bourgoin S, Le Bars D, Artaud F, Clot A M, Bouboutou R, Fournie-Zaluski M C, Roques B P, Hamon M, Cesselin F. Effects of kelatorphan and other peptidase inhibitors on the in vitro and in vivo release of methionine-enkephalin-like material from the rat spinal cord.
J Pharmacol Exp Ther. 1986 Jul.; 238(1):360-6.
7. Browne R G, Segal D S. Behavioral activating effects of opiates and opioid peptides.
Biol Psychiatry. 1980 Feb.; 15(1):77-86.
8. Chen Y, Mestek A, Liu J, Hurley J A, Yu L. Molecular cloning and functional expression of a mu-opioid receptor from rat brain.
Mol Pharmacol. 1993 Jul.; 44(1):8-12.
9. Dauge V, Mauborgne A, Cesselin F, Fournie-Zaluski M C, Roques B P. The dual peptidase inhibitor RB101 induces a long-lasting increase in the extracellular level of Met-enkephalin-like material in the nucleus accumbens of freely moving rats.
J Neurochem. 1996 Sep.; 67(3):1301-8.
10. Di Chiara G, North R A. Neurobiology of opiate abuse.
Trends Pharmacol Sci. 1992 May; 13(5):185-93.
11. Doblman H G, Caron M G, Lefkowitz R J. A family of receptors coupled to guanine nucleotide regulatory proteins.
Biochemistry. 1987 May 19; 26(10):2657-64.
12. Fang L, Knapp R J, Horvath R, Matsunaga T O, Haaseth R C, Hruby V J, Porreca F, Yamamura II. Characterization of [3H]naltrindole binding to delta opioid receptors in mouse brain and mouse vas deferens: evidence for delta opioid receptor heterogeneity.
J Pharmacol Exp Ther. 1994 Feb.; 268(2):836-46.
13. Filliol D, Ghoziand S, Chluba J, Martin M, Matthes H W, Simonin F, Befort K, Gaveriaux-Ruff C, Dierich A, LeMeur M Valverde O, Madonado R, Kieffer B L. Mice deficient for delta- and mu-opioid receptors exhibit opposing alterations of emotional responses.
Nat Genet. 2000 Jun.; 25(2): 195-200.
14. Foumie-Zaluski M C, Coric P, Turcaud S, Lucas E, Noble F, Maldonado R, Roques BP. “Mixed inhibitor-prodrug” as a new approach toward systemically active inhibitors of enkephalin-degrading enzymes.
J Med Chem. 1992 Jun. 26; 35(13):2473-81.
15. Fukuda K, Kato S, Morikawa H, Shoda T, Mori K. Functional coupling of the delta-, mu-, and kappa-opioid receptors to mitogen-activated protein kinase and arachidonate release in Chinese hamster ovary cells.
J Neurochem. 1996 Sep.; 67(3):1309-16.
16. Handa B K, Land A C, Lord J A, Morgan B A, Piance M J, Smith C F. Analogues of beta-LPH61-64 possessing selective agonist activity at mu-opiate receptors.
Eur J. Pharmacol. 1981 Apr. 9; 70(4):531-40.
17. Hescheler J, Rosenthal W, Trautwein W, Schultz G. The GTP-binding protein, Go, regulates neuronal calcium channels.
Nature. 1987 Jan. 29-Feb. 4; 325(6103):445-7.
18. Hughes J, Smith T W, Kosterlitz H W, Fothergill L A, Morgan B A, Morris H R. Identification of two related pentapeptides from the brain with potent opiate agonist activity.
Nature. 1975 Dec. 18; 258(5536):577-80.
19. Jin W, Lee N M, Loh H H, Thayer S A. Opioids mobilize calcium from inositol 1,4,5-trisphosphate-sensitive stores in NG108-15 cells.
J. Neurosci. 1994 Apr.; 14(4):1920-9.
20. Johnson S W, North R A Opioids excite dopamine neurons by hyperpolarization of local interneurons.
J. Neurosci. 1992 Feb.; 12(2):483-8.
21. Kieffer B L, Befort K, Gaveriaux-Ruff C, Hirth C O. The delta-opioid receptor: isolation of a CDNA by expression cloning and pharmacological characterization.
Proc Natl Acad Sci USA. 1992 Dec. 15; 89(24):12048-52. Erratum in: Proc Natl Acad Sci USA 1994 Feb. 1; 91(3):1193.
22. Law P Y, Wong Y H, Loh H H. Molecular mechanisms and regulation of opioid receptor signaling.
Annu Rev Pharmacol Toxicol. 2000; 40:389-430.
23. Le Foil B, Schwartz J C, Sokoloff P. Dopamine D3 receptor agents as potential new medications for drug addiction.
Eur Psychiatry. 2000 Mar.; 15(2):140-6.
24. Maldonado R, Saiardi A, Valverde O, Samad T A, Roques B P, Borrelli E. Absence of opiate rewarding effects in mice lacking dopamine D2 receptors.
Nature. 1997 Aug. 7; 388(6642):586-9.
25. Mansour A, Khachaturian H, Lewis M E, Akil H, Watson S J. Anatomy of CNS opioid receptors.
Trends Neurosci. 1988 Jul.; 11(7):308-14.
26. Matthes H W, Maldonado R, Simonin F, Valverde O, Slowe S, Kitchen I, Befort K, Dierich A, Le Meur M, Dolle P, Tzavara E, Hanoune E, Roques B P, Kieffer B L. Loss of morphine-induced analgesia, reward effect and withdrawal symptoms in mice lacking the mu-opioid-receptor gene.
Nature. 1996 Oct. 31; 383(6603):819-23.
27. Matthews R T, German D C. Electrophysiological evidence for excitation of rat ventral tegmental area dopamine neurons by morphine.
Neuroscience. 1984 Mar.; 11(3):617-25.
28. McBride W J, Murphy J M, Ikemoto S. Localization of brain reinforcement mechanisms: intracranial self-administration and intracranial place-conditioning studies.
Behav Brain Res, 1999 Jun.; 101(2):129-52.
29. Mosberg M I, Hurst R, Hruby V J, Gee K, Yamamura H I, Galligan J J, Burks T F. Bis-penicillamine enkephalins possess highly improved specificity toward delta opioid receptors.
Proc Natl Acad Sci USA. 1983 Oct.; 80(19):587-14.
30. Nieto M M, Wilson J, Cupo A, Roques B P, Noble F. Chronic morphine treatment modulates the extracellular levels of endogenous enkephalins in rat brain structures involved in opiate dependence: a microdialysis study.
J Neurosci. 2002 Feb. 1; 22(3):1034-41.
31. Noble F, Fournie-Zaluski M C, Roques B P. Unlike morphine the endogenous enkephalins protected by RB11 are unable to establish a conditioned place preference in mice.
Eur J. Pharmacol. 1993 Jan. 12; 230(2):139-49.
32. Noble F, Roques B P. Inhibitors of enkephalin catabolism. New therapeutic tool in opioid dependance.
Molecular Biology of addiction, 2000, Humana Press Inc., Totowa, N.J.
33. Noble F. Soleilhac J M, Soroca-Lucas E, Turcaud S, Foumie-Zaluski M C, Roques B P. Inhibition of the enkephalin-metabolizlng enzymes by the first systemically active mixed Inhibitor prodrug RB 101 induces potent analgesic responses in mice and rats.
J Pharmacol Exp Ther. 1992a Apr.; 261(1):181-90.
34. North R A, Williams I T, Surprenant A, Christie M W. Mu and delta receptors belong to a family of receptors that are coupled to potassium channels.
Proc Natl Acad Sci USA. 1987 Aug.; 84(15):5487-91.
35. Olievenstein C. La drogue, 30 ans aprés.
Editions Odile Jacob, 2000.
36. Perrault O, Depoortere R, Morel E, Sanger D J, Scatton B. Psychopharmacological profile of amisulpride: an antipsychotic drug with presynaptic D2/D3 dopamine receptor antagonist activity and limbic selectivity.
J Pharmacol Exp Ther. 1997 Jan.; 280(1):73-82.
37. Pert C B, Snyder S H. Opiate receptor: demonstration in nervous tissue.
Science. 1973 Mar. 9; 179(77):1011-4.
38. Roques B P, Fournie-Zaluski M C. Enkephalin degrading enzyme inhibitors: a physiological way to new analgesics and psychoactive agents.
NIDA Res Monogr. 1986; 70:128-54.
39. Roques BP. Novel approaches to targeting neuropeptide systems.
Trends Pharmacol Sci. 2000 Dec.; 21(12):475-83.
40. Roques BP. Zinc metallopeptidases: active site structure and design of selective and mixed inhibitors: new approaches in the search for analgesics and anti-hypertensives.
Biochem Soc Trans. 1993 Aug.; 21 (Pt 3)(3):678-85.
41. Ruiz-Gayo M, Baamonde A, Turcaud S, Fournie-Zaluski M C, Roques B P. In vivo occupation of mouse brain opioid receptors by endogenous enkephalins: blockade of enkephalin degrading enzymes by RB 101 inhibits [3H]diprenorphine binding.
Brain Res. 1992 Feb. 7; 571(2):306-12.
42. Sharma S K, Klee W A, Nirenberg M. Opiate-dependent modulation of adenylate cyclase.
Proc Natl Acad Sci USA. 1977 Aug.; 74(8):3365-9.
43. Simon E J, Hiller J M, Edelman 1. Stereospecific binding of the potent narcotic analgesic (3H) Etorphine to rat-brain homogenate.
Proc Natl Acad Sci USA. 1973 Jul.; 70(7):1947-9.
44. Spanagel R., Herz A, Shippenberg T S. The effects of opioid peptides on dopamine release in the nucleus accumbens: an in vivo microdialysis study.
J. Neurochem. 1990 Nov.; 55(5): 1734-40.
45. Surprenant A, Shen K Z, North R A, Tatsumi H. Inhibition of calcium currents by noradrenaline, somatostatin and opioids in guinea-pig submucosal neurones,
J Physiol. 1990 Dec.; 431:585-608.
46. Terenius L. Characteristics of the “receptor” for narcotic analgesics in synaptic plasma membrane fraction from rat brain.
Acta Pharmacol Toxicol (Copenh). 1973; 33(5):377-84.
47. Vaccarino A L, Kastin A J. Endogenous opiates: 2000.
Peptides. 2001 Dec.; 22(12):2257-328.
48. Valverde O, Fournie-Zaluski M C, Roques B P, Maldonado R. The CCXB antagonist PD134,308 facilitates rewarding effects of endogenous enkephalins but does not induce place preference in rats.
Psychopharmacology (BerJ). 1996 Jan.; 123(2):119-26.
49. Waksman G, Bouboutoa P, Devin J, Bourgoin S, Cesselin F, Hamon M, Fournie-Zalusli M C, Roques B. In vitro and in vivo effects of kelatorphan on enkephalin metabolism in rodent brain.
Eur J. Pharmacol. 1985 Nov. 5; 117(2):233-43.
50. Yasuda K, Raynor K, Kong H, Breder C D, Takeda J, Reisine T, Bell G I. Cloning and functional comparison of kappa and delta opioid receptors from mouse brain.
Proc Natl Acad Sci USA. 1993 Jul. 15; 90(14):6736-40.
51. Zimmerman D M, Leander J D. Opioid antagonists: structure activity relationships.
NIDA Res Monogr. 1990; 96:50-60.

1. A novel pharmaceutical composition, preferably in kit form, containing a combination of two medicaments intended to be used simultaneously or successively, which consists of a combination of a partial or full antagonist of the dopaminergic receptors and of a prodopaminergic product, as a mixture or in combination with an inert, nontoxic excipient or vehicle suitable for oral, parenteral or transdermal administration.

2. The pharmaceutical composition as claimed in claim 1, in which the dopaminergic receptor antagonist is a D₂ and/or D₃ receptor antagonist.

3. The pharmaceutical composition as claimed in claim 1, in which the dopaminergic antagonist is a D2 and D3 receptor antagonist.

4. The pharmaceutical composition as claimed in claim 1, in which the dopaminergic antagonist is a molecule also having a serotoninergic component.

5. The pharmaceutical composition as claimed in claim 1, in which the dopaminergic antagonist is chosen from amisulpride, risperidone, the D3 antagonist known as SB277 0II-A, sulpiride, metoclopramide and olanzapine.

6. The pharmaceutical composition as claimed in claim 1, in which the dopaminergic antagonist is amisulpride in resolved form and especially S-(−)-amisulpride.

7. The pharmaceutical composition as claimed in claim 1, in which the prodopaminergic product is a substance capable of binding to the opioid receptors or to systems capable of stably exciting the dopaminergic system.

8. The pharmaceutical composition as claimed in claim 7, in which the prodopaminergic product is chosen from methadone, buprenorphine, the product known as LAM, nalorphine, naltrexate and Levallorphan.

9. The pharmaceutical composition as claimed in claim 1, which also contains a neuroleptic.

10. The pharmaceutical composition as claimed in claim 1, in which the combination of dopaminergic antagonist and of prodopaminergic product is in the form of a single defined pharmaceutical composition.

11. The pharmaceutical composition as claimed in claim 1, in which the combination of dopaminergic antagonist and of prodopaminergic product is in the form of a kit containing each of the active principles in a separate form.

12. The pharmaceutical composition as claimed in claim 1, in which the combination of the two active principles is in two identical pharmaceutical forms.

13. The pharmaceutical composition as claimed in claim 1, in which the combination of the two active principles is in two different pharmaceutical forms.

14. The pharmaceutical composition as claimed in claim 1, in which the doses of antidopaminergic substance range from 0.3 to 1200 mg per single intake.

15. The pharmaceutical composition as claimed in claim 14, in which the dose of racemic amisulpride or of amisulpride in the form of the S(−) isomer per single intake ranges from 200 mg to 1200 mg.

16. The pharmaceutical composition as claimed in claim 1, in which the doses of prodopaminergic substance range from 0.2 mg to 300 mg.

17. The pharmaceutical composition as claimed in claim 1, characterized in that it is formed from tablets of amisulpride at a dose of from 100 mg to 400 mg and of tablets of prodopaminergic substance at a dose of from 0.2 to 100 mg per single intake.

18. The pharmaceutical composition as claimed in claim 1, in which the doses of prodopaminergic substances intended for rapid metabolizers are of the order of 200 to 300 mg.

19. The pharmaceutical composition as claimed in claim 1, characterized in that it is in the form of a kit containing two bottles of a solid or liquid preparation of antidopaminergic substance, on the one hand, and of a liquid preparation of prodopaminergic substance, on the other hand.

20. The pharmaceutical composition as claimed in claim 1, consisting of a combination of amisulpride or a salt thereof, in racemic or enantiomerically pure form, and of methadone, characterized in that it contains from 100 to 400 mg of amisulpride and from 0.2 mg to 30 mg of buprenorphine per single intake.

21. The pharmaceutical composition as claimed in claim 1, which is in the form of a kit containing a first pharmaceutically suitable dosage of amisulpride in base form or in salt form, in racemic form or in enantiomeric form, at a dose of from 100 mg to 400 mg, and a second pharmaceutically suitable dosage of methadone containing from 5 to 60 mg per single intake.

22. The pharmaceutical composition as claimed in claim 1, consisting of a combination of risperidone and of a dopaminergic agonist, characterized in that it contains from 1 to 16 mg of risperidone.

23. The pharmaceutical composition as claimed in claim 1, consisting of a combination of amisulpride and of buprenorphine, naltrexone or nalorphine, characterized in that it contains from 400 to 1200 mg of amisulpride and from 0.2 to 30 mg of buprenorphine or naltrexone or nalorphine per single intake.

24. The pharmaceutical composition as claimed in claim 1, intended to be administered at a rate of one to four times a day.

25. A method for combating the different forms of addition to licit or illicit drugs, which consists in administering to an individual displaying addiction phenomena a sufficient and effective amount of a combination of a dopaminergic antagonist and of a dopaminergic agonist, simultaneously or in batch mode, in a single or separate pharmaceutical form.
